(19) United States
(12) Patent Application Publication
(10) Pub. No.: US 2021/0085181 A1
(43) Pub. Date: Mar. 25, 2021

(54) INFORMATION PROCESSING APPARATUS, MEDICAL IMAGING APPARATUS, INFORMATION PROCESSING METHOD, AND RECORDING MEDIUM

(71) Applicants: CANON KABUSHIKI KAISHA, Tokyo (JP); CANON MEDICAL SYSTEMS CORPORATION, Tochigi (JP)

(72) Inventors: Shuhei Toba, Kawasaki-shi (JP); Atsuko Sugiyama, Nasushiobara Tochigi (JP)

(21) Appl. No.: 17/024,101

(22) Filed: Sep. 17, 2020

(30) Foreign Application Priority Data
Sep. 24, 2019 (JP) 2019-173548

(51) Int. Cl. A61B 5/00; G16H 40/20

(52) U.S. Cl. CPC A61B 5/0022; G16H 40/20; A61B 5/0026; A61B 5/002

(57) ABSTRACT

An information processing apparatus, comprising at least one processor; and at least one memory storing a program which, when executed by the processor, causes the information processing apparatus to: detect a specific illness or injury from a medical image taken from a patient; obtain medical worker information including contact information and an attendance status for at least one medical worker related to the detected illness or injury; and notify the medical worker information along with a detection result of the illness or injury.

### BACKGROUND OF THE INVENTION

### Field of the Invention

The present invention relates to an information processing apparatus, a medical imaging apparatus, an information processing method, and a recording medium.

### Description of the Related Art

A medical imaging apparatus may be used to capture images of a body part affected by an illness or injury (disease and trauma) which needs to be addressed urgently or needs to be treated appropriately as soon as possible. Some of such illnesses and injuries are detected as a radiologist interprets such an image. However, other than for urgent examination, it may sometimes take long for the radiologist to interpret the image and notice the condition until the condition becomes serious. Examples of injuries and illnesses which should be treated urgently include “pneumothorax” and “aortic dissection” which are life-threatening if not treated urgently, and “tuberculosis” which may be infectious.

There has been a known technique for automatically detecting such an illness and an injury and providing prompt treatment. Japanese Patent Application Publication No. 2018-182672 discloses a technique for detecting abnormal shadow candidates from medical images. Japanese Patent Application Publication No. 2006-024048 indicates that when an illness or injury which needs to be treated urgently is detected, all the contacts pre-registered in a database are notified.

However, according to the disclosure of Japanese Patent Application Publication No. 2006-024048, all of the pre-registered contacts are notified, some of the registered doctors or radiologists are in the middle of surgery or out of office and cannot be reached as required. Meanwhile, it takes time to find out which doctors can be contacted manually, which can prevent quick treatment.

### SUMMARY OF THE INVENTION

It is an object of the present disclosure to help determine quickly and appropriately a medical worker to be contacted when a specific illness or injury is detected.

One aspect of the disclosure is an information processing apparatus, comprising: at least one processor; and at least one memory storing a program which, when executed by the processor, causes the information processing apparatus to: detect a specific illness or injury from a medical image taken from a patient; obtain medical worker information including contact information and an attendance status for at least one medical worker related to the detected illness or injury; and notify the medical worker information along with a detection result of the illness or injury.

Another aspect of the disclosure is a computer-implemented information processing method, comprising the steps of: obtaining a medical image taken from a patient; detecting a specific illness or injury from the medical image; obtaining medical worker information including contact information and an attendance status for at least one medical worker related to the detected illness or injury; and notifying the medical worker information along with a detection result of the illness or injury.

According to the present disclosure, quick and appropriate communication with a medical worker is achievable when a specific illness or injury is detected.

Further features of the present invention will become apparent from the following description of exemplary embodiments with reference to the attached drawings.

### BRIEF DESCRIPTION OF THE DRAWINGS

FIG. 1 is a view of the configuration of an information processing apparatus according to a first embodiment of the invention;

FIG. 2 is a flowchart for illustrating the entire flow according to the first embodiment;

FIG. 3 is a flowchart for illustrating details of contact information obtaining processing in S203 according to the first embodiment;

FIG. 4 shows the contact information list obtained in S301;

FIG. 5 is a flowchart for illustrating details of working status obtaining processing in S302 according to the first embodiment;

FIG. 6 shows a contact information list obtained in S302;

FIG. 7 shows a contact information list with priorities obtained in S303;

FIG. 8 shows an example of information presented at a display unit according to the first embodiment;

FIG. 9 shows examples of scores and weights to be applied to each item of contact information and a content thereof according to a second embodiment of the invention;

FIG. 10 shows priorities calculated according to the second embodiment; and

FIG. 11 is a view of the configuration of an information processing apparatus according to a fifth embodiment of the invention.

### DESCRIPTION OF THE EMBODIMENTS

First, terms used herein will be described.

An “attending doctor” is a doctor who issues an imaging request with respect to a patient from a hospital information system (HIS) or a radiology information system (RIS). “Medical workers related to an illness or injury” include an attending doctor, a radiologist, a surgeon, a nurse, a clinical technologist, a counselor, and a paramedic (co-medical) who should be contacted by a radiological technologist (hereinafter as the “technologist”) when an illness or injury which needs urgent treatment is found. In the following description, unless otherwise noted, a “medical worker” refers to a medical worker related to an illness or injury.

“Medical worker information” refers to information which is useful in contacting a medical worker and includes the name, the title, the hospital attendance status, and the contact information of a medical worker. Medical worker information may include a contacting priority or the priority level of a contacting method. The “contacting method” refers to how to contact a medical worker, for example by a personal mobile phone, e-mail, an extension in a facility where the worker belongs, and a hospital broadcast. The “contact information” refers to information used to specify a person to be contacted using a certain contacting method, and examples of the information include a telephone number, an e-mail address, an extension number, and a hospital broadcast area (for example a ward name and a room name).

The term “mail” as used herein refers to a system or method for electrically transmitting/receiving a text message or an image in association with a personal account or an organization account (group account) for example in e-mail and short message services, and an instant messaging service. Also, the e-mail address according to the present invention refers to an identifier or a character string used to identify an account for transmitting/receiving a mail. In addition, the “telephone” herein includes any contacting system or method using voice, and the line to be used may be a telephone line or an Internet line, and the telephone also includes a video call.

Now, with reference to the drawings, modes for carrying out the present invention (embodiments) will be described in the following. Note that for example the dimensions, materials, shapes, and relative positions of the components in the following description of embodiments are arbitrary and may be changed depending on the configuration of the device to which the invention is applied or various other conditions. Also, in the drawings, the same or functionally identical elements are designated by the same reference characters.

### First Embodiment

The information processing apparatus 100 according to the embodiment detects an abnormality in an image taken by the medical imaging apparatus and provides information about a doctor to be contacted when an abnormality is detected and information about the contact. This simplifies and quickens the operation of communicating with the doctor by the technologist. In the following example, a specific disease which needs to be urgently treated is detected from a medical image while the detection target may be a trauma instead of the disease.

Configuration

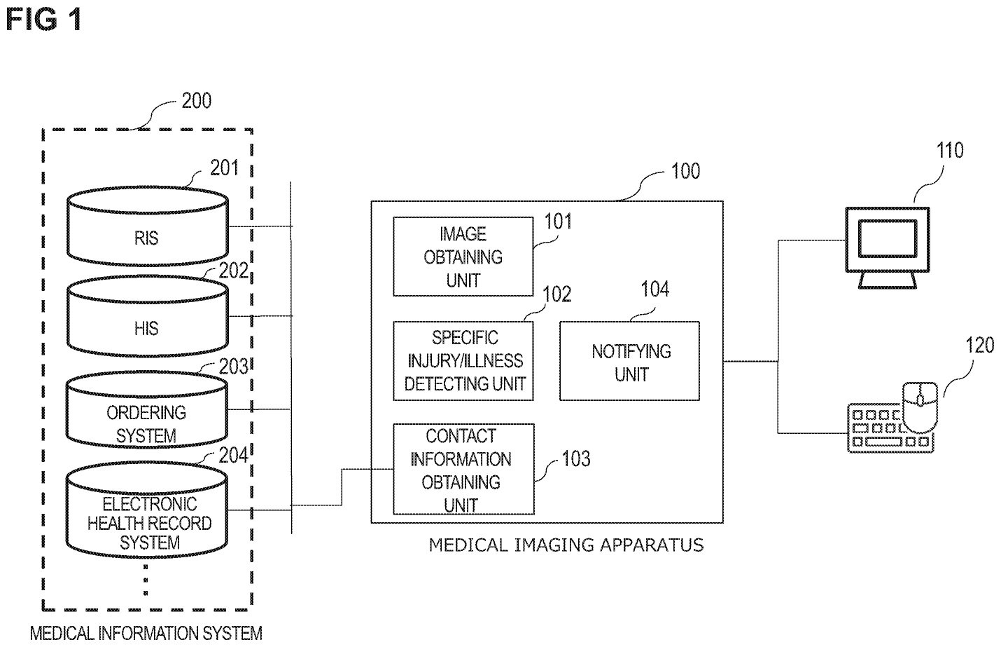

FIG. 1 illustrates the configuration of an information processing apparatus (computer) 100 according to the embodiment. The information processing apparatus 100 according to the embodiment will be described as being incorporated in the console of a medical imaging apparatus such as an X-ray CT system, while the apparatus may be configured as an apparatus different from the imaging apparatus if the apparatus can communicate with the imaging apparatus and obtain medical images.

The information processing apparatus 100 includes an arithmetic processor, a main storage unit, an auxiliary storage unit, and a communication interface. The information processing apparatus 100 is connected with an output device 110 such as a display and an input device 120 such as a keyboard and a mouse device. The information processing apparatus 100 can communicate with a medical information system 200 through a network.

The information processing apparatus 100 is configured to function as an image obtaining unit 101, a specific illness/injury detecting unit 102 (hereinafter also referred to simply as the detecting unit 102), a contact information obtaining unit 103, and a notifying unit 104 as the arithmetic processor executes a computer program. Note that some or all of these functions may be implemented by dedicated hardware.

The image obtaining unit 101 obtains a medical image of a patient from the imaging apparatus. The detecting unit 102 detects a specific illness or injury from the medical image of the patient. Here, examples of the specific illness or injury include diseases which need to be treated urgently such as pneumothorax, aortic dissection, and tuberculosis. The detecting unit 102 includes a model trained for example by machine learning such as deep learning, and the trained model may be used to detect injuries and diseases. The contact information obtaining unit 103 obtains, from the medical information system 200, contact information and hospital attendance status about a medical worker related to the detected illness or injury. The notifying unit 104 notifies the medical worker information obtained by the contact information obtaining unit 103 along with the detection result of the illness or injury. Details of the processing performed by each of the functional parts of the information processing apparatus 100 will be described in the following with reference to the flowcharts.

The medical information system 200 is a general term for information systems in medical facilities, and includes a radiology information system (RIS) 201, a hospital information system (HIS) 202, an ordering system 203, and an electronic health record system 204. Subsystems within the medical information system 200 provide various functions, and the following description includes only exemplary functions.

The RIS 201 is a department information system related to a radiology department within the medical information system 200. The RIS 201 performs management of booking for radiological examination requests, cooperation with examination equipment, examination information management, and cooperation with other department systems. The HIS 202 is configured to improve the efficiency of medical treatment and accounting operations for the entire medical facility and manages information related to medical treatment at the medical facilities and to cooperate with other systems. The information managed by the HIS 202 includes information about patients and staff members (including doctors and technologists). The information about a staff member includes the name, the department, the title of the staff member, the hospital attendance status, the schedule, and the contact of the staff member.

The ordering system 203 generally cooperates with various department systems (such as the RIS and a medical accounting system) using various types of request information for example about radiological examinations, surgeries, and prescriptions from a terminal as order information. In a facility having a system which works within an intra-hospital network including the ordering system 203, an attending doctor registers an examination request in the system, and a technologist takes an image of the patient in response to the request. The examination request information input to the ordering system 203 may include the content of the request (examination content), the time zone, the requesting doctor, the inputter, and patient information. The electronic health record system 204 manages the patient's medical records and shares them with other systems.

Processing

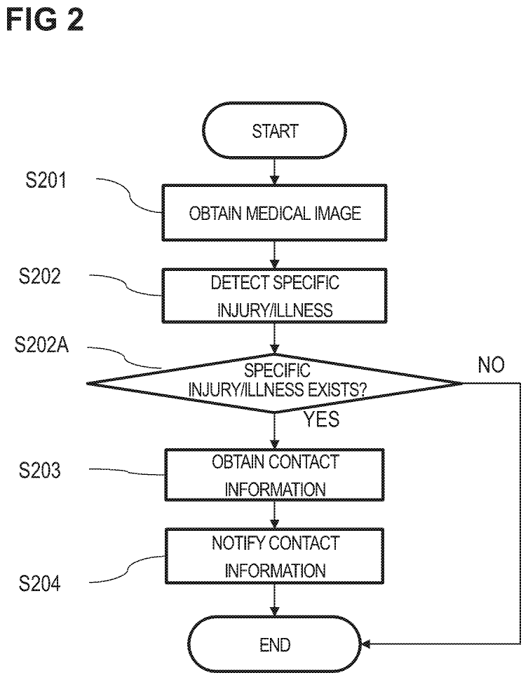

The flow of processing carried out by the information processing apparatus 100 according to the embodiment will be described with reference to the flowchart in FIG. 2.

In step S201, the image obtaining unit 101 obtains a medical image of a patient that has been taken by the technologist. The imaging apparatus includes, for example,

computed radiography (CR) equipment,

computed tomography (CT) equipment,

magnetic resonance imaging (MRI) equipment,

positron emission tomography (PET) equipment,

single photon emission computed tomography (SPECT) equipment, or

ultrasound diagnostic equipment (ultrasound system).

In step S202, the detecting unit 102 detects a specific illness or injury from the medical image obtained by the image obtaining unit 101. The specific illness or injury is, for example, an illness or injury which needs to be treated urgently. The detecting unit 102 detects an illness or injury such as pneumothorax and aortic dissection and outputs for example information about the type of the detected illness or injury and information about the region of the illness or injury in the image. The detecting unit 102 needs only detect any specific illness or injury, and the detection target is not limited to the above-mentioned injuries or diseases. The detecting unit 102 may also use a model trained according to a machine learning algorithm (detection engine) to detect injuries and illnesses, or rule-based processing can be carried out to detect injuries and illnesses.

When no illness or injury is detected in step S202 (NO in S202A), the process ends. Meanwhile, when an illness or injury is detected in step S202 (YES in S202A), the process proceeds to step S203.

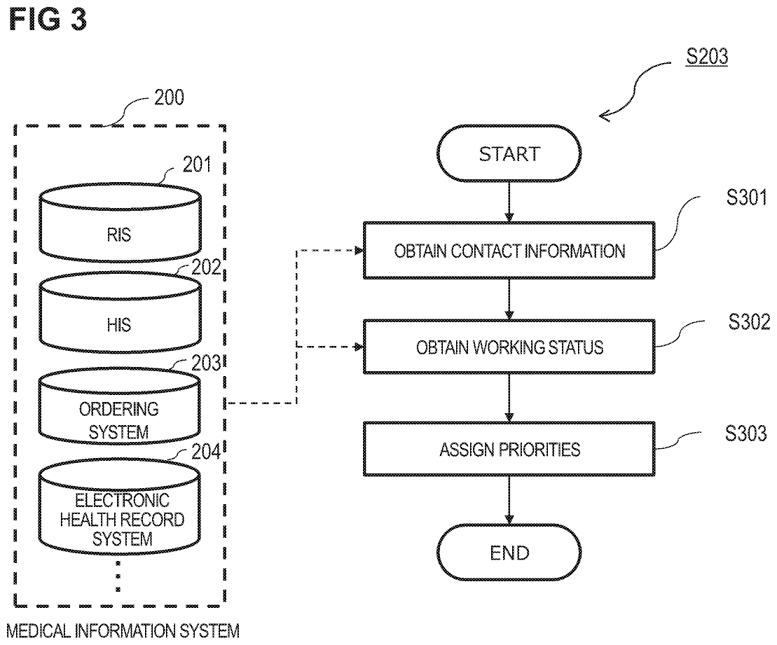

In step S203, when an illness or injury which should be urgently treated is detected by the detecting unit 102, the contact information obtaining unit 103 obtains a contacting method according to the possibility of contacting a medical worker. Hereinafter, with reference to FIG. 3, the flow of the processing by the contact information obtaining unit 103 for obtaining the contacting method will be described.

In step S301, the contact information obtaining unit 103 obtains contact related information about a medical worker related to the illness or injury detected by the detecting unit 102 from the medical information system 200. The medical worker related to the detected illness or injury includes the attending doctor who gave the instruction to take the medical image, the radiologist who interprets the medical image or a surgeon who performs a surgery on the detected illness or injury. Although according to the embodiment, a medical doctor is assumed as a medical worker related to the illness or injury, medical workers related to the illness or injury may include at least one of a nurse, a medical technologist, a counselor, and a paramedic (co-medical).

The contact information obtaining unit 103 obtains information about who the attending doctor is, for example, from the ordering system 203. In a facility which has introduced a system which works within an intra-hospital network such as the ordering system 203, an attending doctor registers a request for examination in the system and the technologist takes an image of the patient in response to the request. The information about the request for examination at this time includes the “requesting doctor,” the “inputter,” the “requested content,” the “time zone.” Therefore, the contact information obtaining unit 103 can specify the “requesting doctor” in the examination request information as an attending doctor. The contact information obtaining unit 103 obtains the pre-registered contact information about the attending doctor from the medical information system 200. The pre-registered contact information about the attending doctor is for example the telephone number of the personal mobile terminal (hereinafter the mobile phone number) and the e-mail address of the attending doctor. The contact information may be obtained from the user management system in the RIS 201 or HIS 202, or may be obtained from any of other related systems such as a personnel system. There may be one or more kinds of information such as phone numbers and e-mail addresses depending on the type of the terminal or the mail service type. In such a case, the contact information obtaining unit 103 stores the contact information separately on a terminal type basis such as a PHS, a smartphone, and a tablet or on a mail service type such as free mail and intra-hospital mail.

Then, the contact information obtaining unit 103 specifies a radiologist who interprets medical images or a surgeon who performs a surgery for the detected illness or injury. The contact information obtaining unit 103 obtains all of the mobile numbers and mail addresses pre-registered in the medical information system 200 as contact information for the specified radiologist and surgeon. A radiologists and a surgeon can be extracted by previous filtering according to the kind of the detected illness or injury and the expertise of the medical facilities or doctors. For example, when the detected illness or injury is pneumothorax, contact information only about a radiologist (interpreting doctor) who specializes in interpreting the chest and a respiratory surgeon may be obtained. The contact information may be obtained from the RIS 201 or HIS 202 user management system as well as from the attending physician's contact information, or from other relevant systems, such as the personnel system.

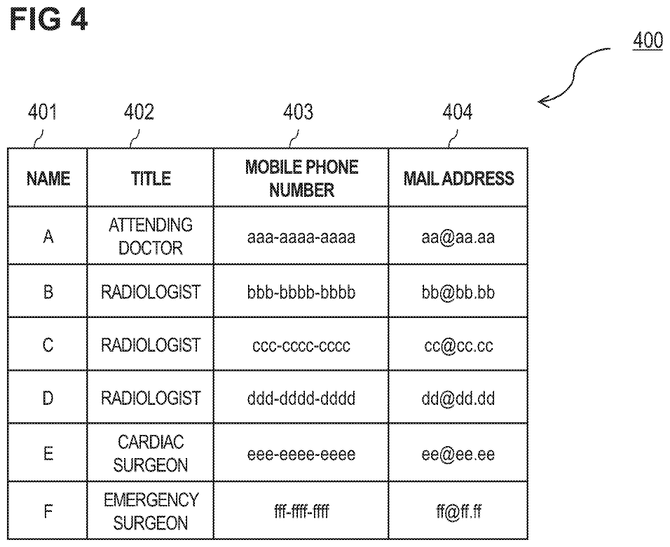

By the above step S301, a list 400 including the contact information about the doctor including the information shown in FIG. 4 is obtained. The list 400 includes the name 401, the title 402, the mobile phone number 403, and the e-mail address 404 of each of doctors. Here, the list 400 includes one mobile number and one e-mail address, but the list may include more than one piece of such contact information. In the following description, a doctor contact information list including the contact information such as mobile phone numbers and e-mail addresses will be simply referred to as a contact information list.

The list 400 obtained in step S301 includes contact information about doctors to be contacted at the time of the discovery of a specific illness or injury, while a doctor is not always reachable when the doctor is not present or in the middle of a surgery.

Therefore, in step S302, the contact information obtaining unit 103 obtains, as information about contacting possibility, the working statuses of medical workers from the medical information system 200. More specifically, the contact information obtaining unit 103 extracts a working status on the basis of the request information obtained from the ordering system 203 in the medical information system 200.

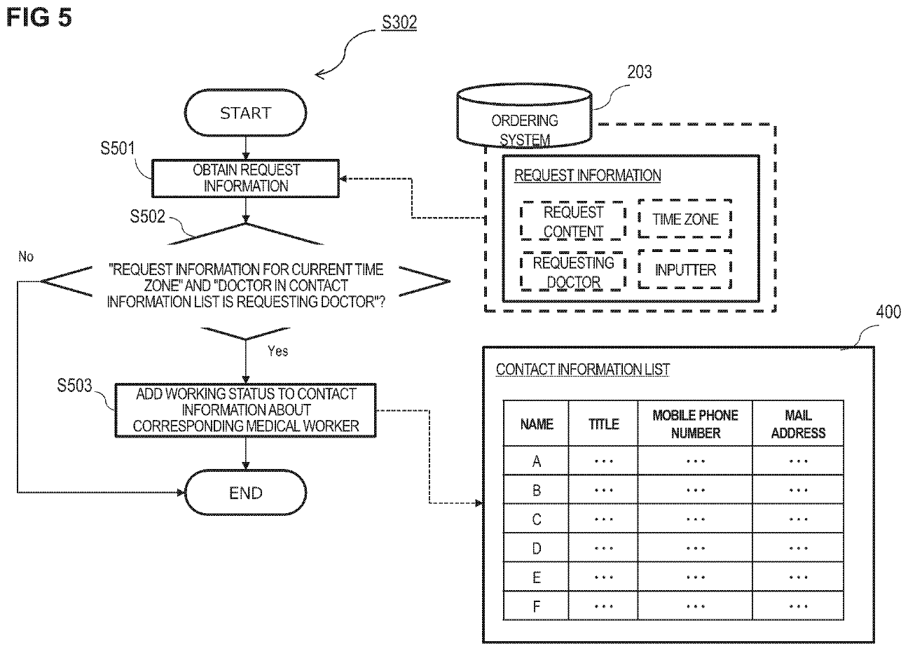

Here, the working status can be obtained for example by using request information in the ordering system 203 by the flow shown in FIG. 5. In step S501, the contact information obtaining unit 103 can obtain the request information from the ordering system 203. The request information includes the “content of request” for examination, “time zone,” the “requesting doctor,” and the “inputter.”

In step S502, the contact information obtaining unit 103 extracts request information indicating that the “time zone” is the current time zone and the “requesting doctor” is a doctor included in the contact information list 400. The “requested content” of the requested information includes information such as “radiological examination” and “surgery.” Therefore, in step S301, the contact information obtaining unit 103 can obtain the working status of the doctor such as “examining,” “surgery,” “interpreting,” “consulting,” and “interviewing,” for each of the doctors whose contact information is obtained in step S301 by comparing the name of the attending doctor and the time zone with those of the doctor. In addition, since the request information includes information about a corresponding facility and equipment, and when an extension number of the facility is registered in the medical information system 200, the contact information obtaining unit 103 can obtain the extension number from the medical information system as the doctor's contact information.

In step S503, the contact information obtaining unit 103 adds, to the list 400 produced in step S301, information including the working status of each of the doctors. In addition, when the facility where the doctor is located and its extension number are obtained, the contact information obtaining unit 103 adds the facility (room) and its extension number to list 400.

The working status of the doctor may be obtained from the medical information system 200 other than the ordering system 203. For example, when there is no request information indicating the corresponding doctor is the requesting doctor in the ordering system 203, the contact information obtaining unit 103 may obtain the contact information of the doctors from the scheduling system, which manages the schedules of the doctors.

In addition, when there is no request information indicating that the doctor who corresponds to the request information in the ordering system 203 is the requesting doctor, the contact information obtaining unit 103 works with an attendance system which manages attendance, and the “attendance status” may be obtained. For example, when there is no record of attendance at the hospital on the date of imaging of the patient, the working status is simply “absent” and when there is an attendance record and no departure record, the working status is simply referred to as “present.” The hospital attendance status indicates whether the doctor is present in the medical facility, and when the doctor is present in the medical facility, the whereabouts of the doctor may be included. The working status can be considered as a specific example of the hospital attendance status.

The contact information obtaining unit 103 uses a system other than the ordering system 203 to determine where each of the doctors is currently present (the current location). For example, a doctor may perform an emergency surgery which is not scheduled, or the surgery may be completed early or conversely may take longer than planned. Therefore, the contact information obtaining unit 103 can specify the current location of each of the doctors in addition to or in place of the location obtained from the ordering system 203. The current location of a doctor may be determined by a room entrance/exit management system or positional information about a personal mobile terminal or a communication history.

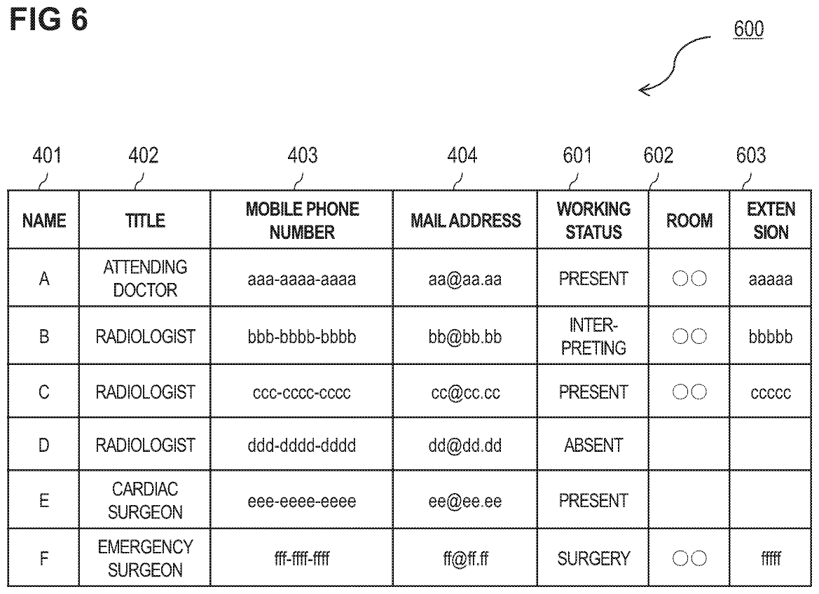

FIG. 6 shows an exemplary contact information list 600 with working statuses and facility extension numbers added by the above processing. The list 600 shows the working status 601 of each of the doctors with respect to the list 400 obtained in step S301. When the facility (room) where the doctor is present is known, the name of the facility 602 and the extension number 603 are added.

In steps S301 and S302, information about the working statuses and the facility extension numbers of the attending doctor and a plurality of radiologists or surgeons are added to the contact information list, and the technologist can be aware of whether each of the doctors can be contacted. However, simply presenting the contact information list 600 is not enough and it may be difficult detect for the technologist to determine who should be contacted first when an illness or injury which needs to be treated urgently is detected.

Therefore, in step S303, the contact information obtaining unit 103 sets a priority for each contact target (medical workers) in the contact information list depending on the title, the hospital attendance status and, the obtained contact information of the medical worker. In this way, the technologist can be aware of which medical worker has a high priority. If the priority information is displayed in a way which allows the technologist to determine the priority of a contact target in the contact information list, the information may be displayed in the form of a character string, symbols, or numbers, or a combination of the above.

In the following example, the information indicating priorities is presented in descending order of priorities defined as “essential contact” “possible contact candidate,” “contact reserve candidate,” and “unreachable.” Since the attending doctor who has requested for imaging should be contacted first, the doctor is indicated as the “essential contact.” Then, a radiologist or a surgeon is assigned with the next highest priority depending on the working status and the presence/absence of the contact information. As for the working status of the radiologists, the “consulting,” the “interviewing,” the “examining,” the “surgery,” the “present,” and the “absent” are indicated, the doctor in the midst of “surgery” cannot be contacted and therefore the doctor is indicated as “unreachable.” The “mobile phone number,” the “e-mail address,” and the “extension number” are listed as emergency contact information, but doctors who do not have appropriate contact information available as a method for contacting in an emergency are indicated as “contact reserve candidates.” The appropriate method for contacting in an emergency may be a telephone call to a personal mobile terminal and a telephone call to the facility's extension number, and the appropriate contact information in an emergency as a method for contacting in an emergency is a “mobile phone number” and an “extension number.” In addition, doctors whose working status are “absent” are not likely to be contacted, and are therefore considered as “contact reserve candidates”. The remaining doctors have a working status of “consulting,” “interviewing,” “examining,” or “present,” and have their “mobile phone number” or “extension” information. The priorities for these doctors are indicated as “possible contact candidates.”

Here, an example of the priority determination rule is illustrated, while specifically what kind of priority is set for doctors in which status may be defined as appropriate, and may be changed depending on the operation by the medical facility operating the system according to the embodiment. The priority determination rule is stored in advance in the information processing apparatus 100 as setting information, and the contact information obtaining unit 103 can determine the priority for each of the doctors by referring to the setting information.

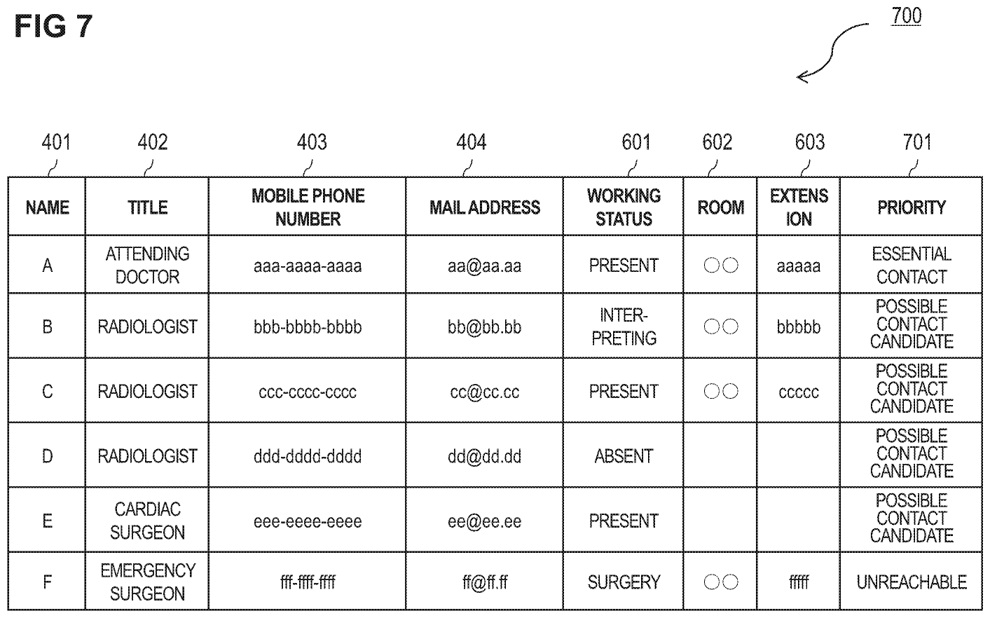

FIG. 7 shows a contact information list 700 which includes a priority 701 assigned by the processing in step S303. By the above described processing, the contact information obtaining processing in step S203 ends. The contact information list 700 in FIG. 7 can be considered as the result of the contact information obtaining processing in step S203.

In step S204, the notifying unit 104 presents the contact information list 700 along with the emergency injury/disease detection result by the detecting unit 102 to the technologist. At the time, the notifying unit 104 may present the medical worker information for medical workers whose priorities satisfy the criteria for notification on the basis of the priorities assigned in step S303. For example, the notifying unit 104 may only present information about a doctor whose priorities are the “essential contact” and the “reachable candidate,” and does not have to indicate information about doctors whose priorities are the “contact reserve candidate” and the “unreachable.” In addition, the notifying unit 104 may present information related to contacts only for a predetermined number of doctors with high priorities.

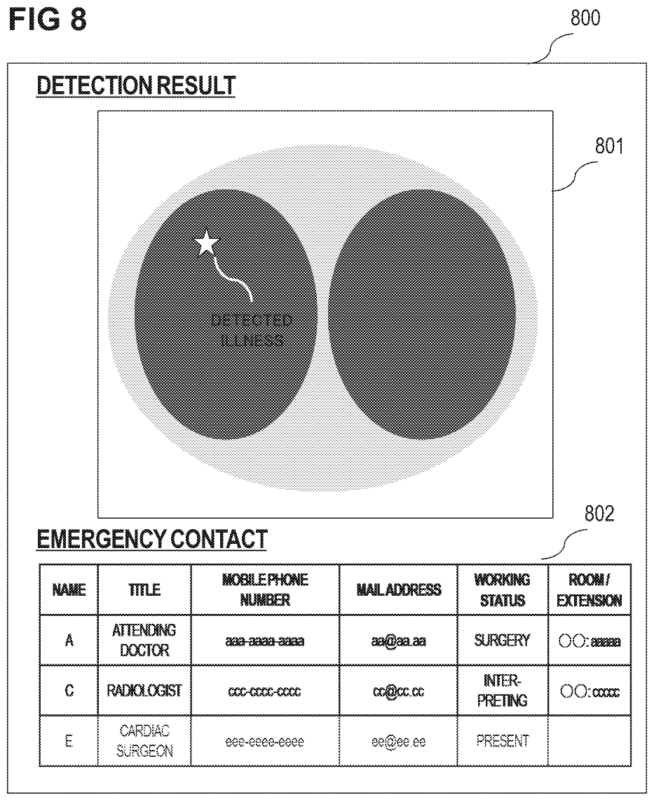

FIG. 8 is an example of a notification screen 800 by the notifying unit 104. The notification screen 800 includes a detection result 801 about an illness or injury which needs to be treated urgently and a contact information group (medical worker information group) 802 obtained by excluding “contact reserve candidates” and “unreachable” for the priority 701 from the contact information list 700. The medical worker information presented by the notifying unit 104 includes the name, title, the hospital attendance status, the contact information, and information indicating the contact priority of each of the medical workers. The medical worker information includes, as contact information, any of a personal mobile terminal number, an e-mail address, the extension number of the facility where the worker is present.

As described above, the information processing apparatus according to the embodiment is triggered by the detection of an illness or injury which needs to be treated urgently to notify contact information about medical workers related to the illness or injury along with the detection result. Therefore, when an emergency illness or injury is detected, contact information about the medical workers who should be contacted is immediately available to the technologist. In addition, the notified contact information is accompanied by information on contact availability (working status), so that the technologist can be aware of the working statuses of the doctors and contact, as appropriate, any of the reachable doctors.

### Second Embodiment

According to the embodiment, the priority assigning processing (step S303) is different from the first embodiment. The other features and processing according to the embodiment are identical to those according to the first embodiment, so the description thereof will not be provided.

According to the embodiment, in the priority assignment processing in step S303, each item of contact information is provided with a weight or a score as a parameter and the priority level is calculated. In this way, emergency contact information to be presented can be narrowed down in a more flexible manner depending on the facility's situation.

According to the embodiment, the contact information obtaining unit 103 calculates a score for each item for each of medical workers included in the medical worker information (contact information list) and adds up the scores according to the weights of the items to obtain a priority for the medical worker. Specifically, the contact information obtaining unit 103 obtains a score for each of the title, the hospital attendance status, and the obtained contact information of the medical worker, and sets a priority on the basis of the weighted sum of the scores. In this way, according to the embodiment, the numerical value assigned to the content of each item for each medical worker is referred to as “score,” the number assigned to each item in emergency contacting according to its importance is referred to as “weight.”

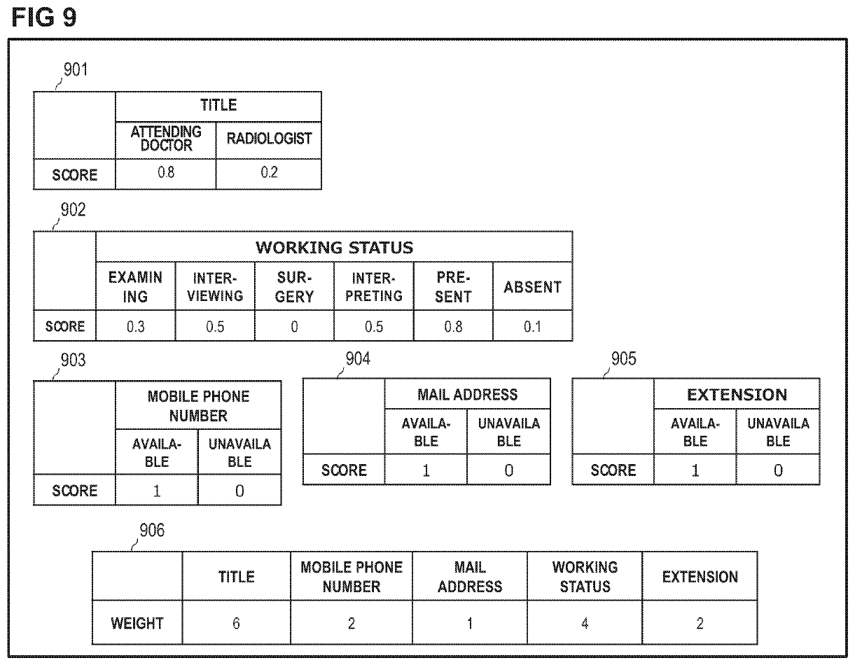

FIG. 9 shows a table including scores and weights. Scores 901 to 905 are numerical values assigned to the contents of the items, the “title,” the “working status,” the “mobile phone number,” the “e-mail address,” and the “extension number,” respectively. Scores 903 to 905 for the items which are not divided into categories such as the “mobile phone number” and the “extension number” each take a binary value depending on the presence and absence of information. Meanwhile, the scores 901 and 902 for items which can be categorized such as the title and working status each take a numerical value according to the presence/absence of information. For example, the score of the “attending doctor” is 0.8, the score of the “radiologist” is 0.2, so that the score of the attending doctor is set to a greater value than the other, indicating the importance of information about the attending doctor. The same applies to the scores for each of the contents of the other items.

A weight 906 represents the degree of how important each item's score is in calculating a priority for each contact target (doctor). In the illustrated example, a maximum weight is given to the “title” and the next largest weight is given to the “working status,” indicting that the weight for “title” is given the highest weight indicating that the degree of the importance of these items is high. The weight for the “mobile phone number” and the “extension number” is 2, the weight for the “mail address” is 1, indicating that the telephone is given more importance than mailing as means for contacting in an emergency.

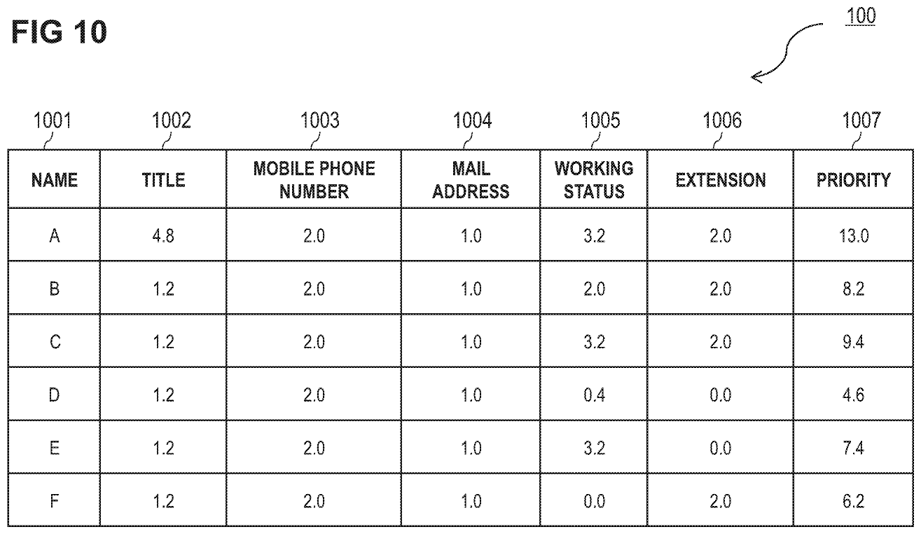

FIG. 10 shows weighted scores 1002 to 1006 for the items and a priority 1007 as the sum of the weighted scores corresponding to the contact information list 600 (FIG. 6) obtained in step S302. For example, for doctor “A,” the score 1002 is 0.8 because the “title” of the doctor is the attending doctor, and since the weight for “title” is 6, the score 1002 for the weighed score for the “title” of doctor “A” is 4.8. Similarly, the weighted scores for “mobile phone number,” the “e-mail address,” the “working status,” and the “extension number” are 2.0, 1.0, 0.8, and 2.0, respectively. The priority 1007 for the doctor “A” is obtained as 13.0 which is the sum of the weighting scores 1002 to 1006. The same applies to the other doctors (contact targets).

According to the embodiment, in step S204, the notifying unit 104 notifies medical worker information about a medical worker whose priority meets the criteria. For example, the notifying unit 104 notifies contact information about a predetermined number of persons (e.g., three persons) starting from the person with the higher priority along with the detection result of the illness or injury. The notifying unit 104 may notify contact information about a medical worker whose priority is not less than a threshold. The threshold may be determined according to the urgency of the illness or injury detected by the detecting unit 102 or may be set to a predetermined value.

As described above, according to the embodiment, weights and scores as parameters are set for contact information, and the priority is calculated, so that the information may be narrowed down in a more flexible manner depending on the state of the facility.

### Third Embodiment

According to the embodiment, the priority assigning processing (step S303) is different from the first embodiment. The other features and processing according to the embodiment are identical to those according to the first embodiment, and therefore the description will not be provided.

According to the embodiment, contact information about a radiologist who has previously interpreted the medical images of the patient is presented with a priority.

The processing flow according to the embodiment up to step S202 shown in FIG. 2 is identical to the described processing flow according to the first embodiment, and therefore, the description up to the step is skipped and the processing after the step will be described.

According to the embodiment, in step S301 shown in FIG. 3, the contact information obtaining unit 103 obtains, from the medical information system, information about whether a medical worker has an interpretation history about an image-taken patient in obtaining contact information about the medical worker. The information about whether there is an interpretation history will be also referred to as interpretation history information. For example, the interpretation history information is information about “whether a radiologist has interpreted an image of a patient in the past.” The record of past interpretation of the image of the patient can be obtained from a cooperating medical information system such as the electronic health record system 204 and the RIS 201 through a network in a facility where the medical worker belongs. The contact information obtaining unit 103 refers to the interpretation history related to the past examination of the image-taken patient, and adds information indicating “with an interpretation history,” to the contact information about any radiologist corresponding to the person who has carried out the interpretation. The contact information obtaining unit 103 adds information indicating that “no interpretation history” to the contact information about the other radiologists.

In step S204, the notifying unit 104 presents information indicating the presence/absence of an interpretation history along with along with the medical worker information. Specifically, the notifying unit 104 presents a contact information list including the interpretation history information on the notification screen 800 (FIG. 8).

As described above, according to the embodiment, the contact information, including information about the added interpretation history, can be assigned a priority, so that contact information about a more suitable radiologist can be presented with a higher priority.

In the above description, the embodiment has been described as a modification of the first embodiment, while the embodiment may be combined with the second embodiment. When the embodiment is combined with the second embodiment, a score corresponding to a content and a weight for the item score are defined for the “interpretation history.” Then, the contact information obtaining unit 103 determines a priority as a weighted sum of the scores for multiple items including the interpretation history. In this case, the contact information obtaining unit 103 determines a priority for the medical worker with an interpretation history to be higher than a priority for a medical worker without such an interpretation history. In this way, priorities can still be assigned in consideration of interpretation histories by radiologists.

### Fourth Embodiment

While according to the first to third embodiments, all the obtained contacting methods are presented, the notifying unit 104 notifies only contact information related to some of contacting methods included in the medical worker information. Therefore, according to the embodiment, the contacting methods are presented with priorities, and information to be presented is switched depending on the priorities.

The processing until the contact information is obtained and priorities are assigned to the contact targets is identical to that in the described processing flow according to the first to third embodiments (steps S201 to S203). In step S204, the notifying unit 104 similarly presents only contact targets (doctors) with higher priorities using the output device 110. According to the embodiment, in step S204, the notifying unit 104 presents only contacting methods with higher priorities instead of presenting contact information (such as a mobile phone number and an e-mail address) related to all the methods for contacting contact targets.

There are several possible methods for assigning a priority to a contacting method. For example, the notifying unit 104 assigns a numerical value representing the importance of each of the contacting methods in advance, and only contacting methods (contact information) with degrees of importance higher than a threshold may be presented. Here, the weight 806 (FIG. 8) for each item in the above description of the second embodiment may be used to represent a degree of importance about each of the methods or any other value may be set. The threshold may be fixed for the entire system or may vary depending on the situation. When the weight 806 is used to represent a degree of importance about each contacting method and a threshold value of at least 2 is set, only the “mobile phone number” and “extension number” with a weight of at least 2 are presented in step S204.

The doctors for whom high-priority contacting methods are presented may be limited to doctors other than the attending doctor or may be all the doctors. Alternatively, for doctors whose priority as a contact target is equal to or greater than the threshold, all the contacting methods are presented, and for doctors whose priority as a contact target is less than the threshold, only some of the contacting methods may be presented.

The notifying unit 104 may exclude a doctor who has no information about a contacting method with a degree of importance (weight) being equal to or greater than the threshold regardless of the priority degree of the contacting method. Alternatively, for a doctor for whom no information is available about any contacting method with a degree of importance (weight) being equal to or greater than the threshold, the notifying unit 104 may present information about the contacting method with the highest degree of importance among the contacting methods about which the information is available.

A priority about a contacting method may be assigned on the basis of the urgency of a detected illness or injury. The specific illness/injury detecting unit 102 determines not only the presence or absence of an illness or injury, but also the urgency of the illness or injury. The urgency of the illness or injury may be determined according to a guideline for the type of illness or injury using the type of the detected illness or injury and the region information. For example, if the type of illness or injury is “pneumothorax”, the severity of the pneumothorax is determined on the basis of the following criteria.

Mild pneumothorax: the apex of the lung is above the clavicle.

Moderate pneumothorax: the apex of the lung is below the clavicle and the lung volume is not less than 50% of the total unilateral volume.

Severe pneumothorax: collapse is observed and the lung volume is not more than 50% of the total unilateral volume.

The specific illness/injury detecting unit 102 classifies the emergencies of illnesses or injuries from region information about the detected illness or injury according to the diagnostic criteria.

The information processing apparatus 100 according to the embodiment pre-stores combinations of emergencies of illnesses or injuries and contacting methods (contact information). For example, the information processing apparatus 100 according to the embodiment pre-stores such a rule that “if the classification result indicates severe pneumothorax, information about the mobile phone number and extension number” is presented, “If the classification result indicates mild or moderate pneumothorax, the extension number, and e-mail address information are presented.” Then, in step S204, the notifying unit 104 presents a contacting method according to the urgency of the illness or injury from the output device 110.

Instead of establishing a rule for combination of the urgency of the illness or injury and a contacting method to be presented, the threshold for the importance (weight) of the contacting methods may be switched according to the urgency of the illness or injury. For example, when the threshold is lowered as the urgency of an illness or injury increases, so that more contacting methods are presented.

As described above, according to the embodiment, an appropriate contacting method can be presented switchably according to the status of the facility and the condition of the illness or injury. In this way, in the case with higher urgency among detected urgent illnesses and injuries, such a contacting method can be presented that it can be confirmed that a doctor is notified of a contact content.

### Fifth Embodiment

According to the first to fourth embodiments, information about emergency contact is presented to the output device 110, but according to the embodiment, the emergency contacts are automatically contacted at the same time as the information is displayed on the output device 110. Specifically, the notifying unit 104 notifies at least some of medical workers using at least one of the telephone line to which the information processing apparatus is connected, the mail service system, the hospital broadcast system in a facility where a medical worker belongs.

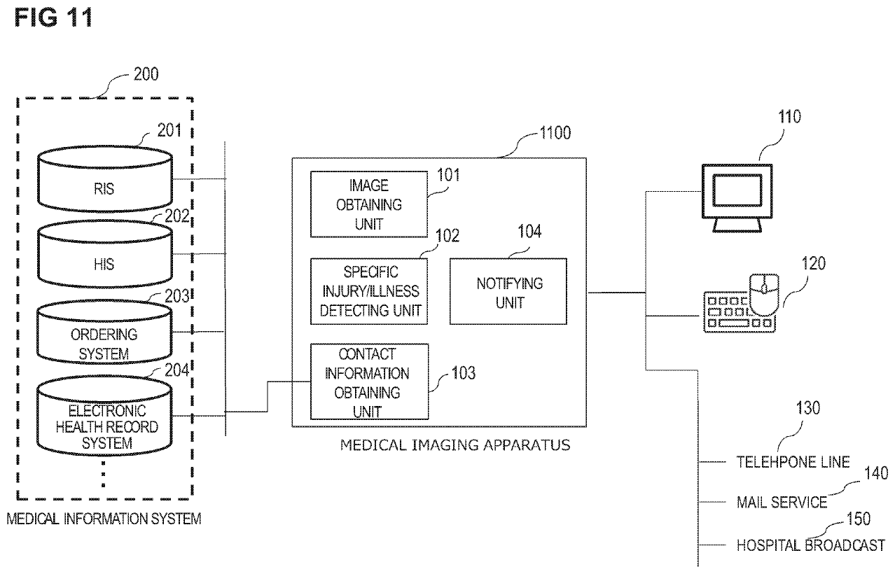

FIG. 11 shows the configuration of an information processing apparatus 1100 according to the embodiment. The features identical to the embodiments are designated by the same reference characters and the description will not be provided. The information processing apparatus 1100 according to the embodiment has a microphone and a speaker for input and output of an operator's voice and is connected with a mobile terminal such as a PHS and a mobile phone, a telephone line 130 to the extension in the facility, the mail service 140 or the hospital broadcast system 150.

The processing flow according to the embodiment is identical to the processing flow according to the first embodiment up to step S203 shown in FIG. 2, the description is skipped and the processing after the step will be described. In step S204, the notifying unit 104 automatically starts a call or sends a message to the contact of the doctor with the highest priority in the contact information obtained by the contact information obtaining unit 103. In this case, which contacting method is to be used by the notifying unit 104 for contacting can be set in advance.

The contact to be automatically contacted can also be switched in consideration of the priorities according to the second to fourth embodiments. For example, a parameter may be set in advance so that contacts with a value of priority 1007 of at least 8 in FIG. 10 are contacted automatically.

As in the forgoing, according to the embodiments, the procedure by the technologist to “check presented emergency contacts and make contact with one of the doctors” can be eliminated, so that the technologist can make contact with the doctor more appropriately.

### OTHER EMBODIMENTS

The present invention can be implemented by supplying a program which implements at least one of the functions according to the embodiments to a system or a device through a network or a recording medium, and allowing at least one processor in the computer of the system or the device to read out and execute the program. The present invention can be carried by a circuit which implements at least one function (such as an ASIC).

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2019-173548, filed on Sep. 24, 2019, which is hereby incorporated by reference herein in its entirety.

What is claimed is:

1. An information processing apparatus, comprising:
at least one processor; and
at least one memory storing a program which, when executed by the processor, causes the information processing apparatus to:
detect a specific illness or injury from a medical image taken from a patient;
obtain medical worker information including contact information and an attendance status for at least one medical worker related to the detected illness or injury; and
notify the medical worker information along with a detection result of the illness or injury.

2. The information processing apparatus according to claim 1, wherein the at least one medical worker related to the detected illness or injury is an attending doctor who has given an instruction to take the medical image, a radiologist who interprets the medical image, or a surgeon who performs surgery to the detected illness or injury.

3. The information processing apparatus according to claim 1,
wherein the program causes the information processing apparatus to set a contact priority for each of the at least one medical worker depending on a title and the attendance status of the at least one medical worker, and
wherein notifying the medical worker information comprises notifying the medical worker information about a medical worker with the priority which satisfies a criteria.

4. The information processing apparatus according to claim 1, wherein the program causes the information processing apparatus to set a contact priority for each of the at least one medical worker depending on a title, the attendance status, and the obtained contact information of the at least one medical worker, and
wherein notifying the medical worker information comprises notifying the medical worker information about a medical worker with the priority which satisfies a criteria.

5. The information processing apparatus according to claim 4,
wherein obtaining the medical worker information comprises obtaining scores for all of the title, the attendance status, and the obtained contact information of the at least one medical worker, and setting the priority based on a weighted sum of the scores, and
wherein notifying the medical worker information comprises notifying the medical worker information about a medical worker with a value of the priority equal to or greater than a threshold.

6. The information processing apparatus according to claim 5, wherein the threshold is determined according to urgency of the detected illness or injury.

7. The information processing apparatus according to claim 1,
wherein the program causes the information processing apparatus to set a contact priority for each of the at least one medical worker, and
wherein the notified medical worker information includes information indicating the priority.

8. The information processing apparatus according to claim 1,
wherein obtaining the medical worker information comprises obtaining information about whether there is an interpretation history related to the image-taken patient by the at least one medical worker from a cooperating medical information system through a network in a facility where the at least one medical worker belongs, and
notifying the medical worker information comprises presenting information indicating presence/absence of the interpretation history along with the medical worker information.

9. The information processing apparatus according to claim 1,
wherein obtaining the medical worker information comprises obtaining information about whether there is an interpretation history related to the image-taken patient by the at least one medical worker from a cooperating medical information system through a network in a facility where the at least one medical worker belongs, and setting a higher priority for a medical worker having the interpretation history than a medical worker without the interpretation history.

10. The information processing apparatus according to claim 1, wherein the attendance status is information related to a working status or a location of the at least one medical worker extracted from a cooperating medical information system through a network in a facility where the at least one medical worker belongs.

11. The information processing apparatus according to claim 10, wherein obtaining the medical worker information comprises extracting the working status based on request information obtained from an ordering system included in the medical information system.

12. The information processing apparatus according to claim 1, wherein the contact information includes at least one of a telephone number of a personal mobile terminal, a mail address, a telephone number of an extension in a facility where the at least one medical worker is located.

13. The information processing apparatus according to claim 1, wherein obtaining the medical worker information comprises extracting the contact information from a cooperating medical information system through a network in a facility where the at least one medical worker belongs.

14. The information processing apparatus according to claim 1, wherein notifying the medical worker information comprises notifying contact information related to some of contacting methods in the contact information included in the medical worker information.

15. The information processing apparatus according to claim 14, wherein the program causes the information processing apparatus to determine which contacting method-related contact information is to be notified, according to urgency of the detected illness or injury.

16. The information processing apparatus according to claim 1, wherein notifying the medical worker information comprises notifying at least one of the at least one medical worker using at least one of a telephone line, a mail service system, and a broadcast system in a facility where the at least one medical worker belongs.

17. The information processing apparatus according to claim 1, wherein the program causes the information processing apparatus to obtain the medical image taken from the patient from a medical imaging apparatus.

18. A medical imaging apparatus, comprising:
an imaging unit which takes a medical image of a patient; and
the information processing apparatus according to claim 1 which carries out notification based on the medical image taken by the imaging unit.

19. A computer-implemented information processing method, comprising the steps of:
obtaining a medical image taken from a patient;
detecting a specific illness or injury from the medical image;
obtaining medical worker information including contact information and an attendance status for at least one medical worker related to the detected illness or injury; and
notifying the medical worker information along with a detection result of the illness or injury.

20. A non-transitory computer-readable medium storing a program, which, when executed by a computer, causes the computer to execute:
a step of obtaining a medical image taken from a patient;
a step of detecting a specific illness or injury from the medical image;
a step of obtaining medical worker information including contact information and an attendance status for at least one medical worker related to the detected illness or injury; and
a step of notifying the medical worker information along with a detection result of the illness or injury.
